(19) United States
(12) Patent Application Publication
(10) Pub. No.: US 2014/0088562 A1
(43) Pub. Date: Mar. 27, 2014

(54) SYSTEM AND METHOD OF PRE-AORTIC GANGLION ABLATION

(71) Applicants: Denise Barbut, New York, NY (US); Allan Rozenberg, San Diego, CA (US); Axel Heinemann, New York, NY (US)

(72) Inventors: Denise Barbut, New York, NY (US); Allan Rozenberg, San Diego, CA (US); Axel Heinemann, New York, NY (US)

(21) Appl. No.: 14/071,136

(22) Filed: Nov. 4, 2013

Related U.S. Application Data
(63) Continuation-in-part of application No. 13/787,325, filed on Mar. 6, 2013.
(60) Provisional application No. 61/641,599, filed on May 2, 2012.
(60) Provisional application No. 61/724,086, filed on Nov. 8, 2012.
(60) Provisional application No. 61/733,034, filed on Dec. 4, 2012.
(60) Provisional application No. 61/739,396, filed on Dec. 19, 2012.

(51) Int. Cl. A61B 18/14; A61B 18/02; A61B 17/34

(52) U.S. Cl. CPC A61B 18/1492; A61B 17/3401; A61B 18/02; A61B 17/3421
USPC 604/512; 606/41; 606/34; 606/33; 604/506; 606/20

(57) ABSTRACT

A method of modulating a physiological parameter of a patient is provided. The method includes disabling one or more pre-aortic ganglion cells within a pre-aortic ganglion and improving the physiological parameter. The method further includes destroying a pre-aortic ganglion cell to prevent regeneration.

This application is a continuation-in-part of U.S. application Ser. No. 13/787,325 filed Mar. 6, 2013, which is pending and claims priority to U.S. Ser. No.: 61/641,599, filed on May 2, 2012, and U.S. Ser. No.: 61/724,086, filed on Nov. 8, 2012, and U.S. Ser. No.: 61/733,034, filed on Dec. 4, 2012, and U.S. Ser. No.: 61/739,396, filed on Dec. 19, 2012, the entireties of which are incorporated herein by reference.

### FIELD OF THE INVENTION

The present invention relates generally to the field of hypertension. More specifically, the present invention relates to a system and method of pre-aortic ganglion ablation for the treatment of hypertension.

### BACKGROUND OF THE INVENTION

Hypertension affects tens of millions of individuals. Untreated hypertension is associated with stroke, heart failure and renal failure. Most patients with hypertension are currently treated pharmacologically, many with multiple medications. A quarter of these patients are resistant to medication and their blood pressure poorly controlled, putting them at added risk for complications.

Activation of the sympathetic nervous system is thought to play a significant role in exacerbating hypertension in the later stages of the disease. Reducing such sympathetic activation has been shown to reduce blood pressure in these circumstances.

Recently, mechanical ablation of the renal nerves surrounding the renal artery has been shown to reduce blood pressure in patients with resistant hypertension. The technique consists of an endovascular, arterial procedure and involves ablation of post-ganglionic sympathetic nerve fibres, accessed through the wall of the renal arteries bilaterally. Renal artery denervation, as the procedure is known, has been shown to reduce systolic and diastolic pressures by up to 20 mm and 10 mm respectively, and to be persistent out to a year following the procedure. The incidence and severity of procedure related and late complications are as yet unknown, as is the long term benefit of blood pressure. Renal nerve fibres regenerate and the hypotensive effect of this ablative procedure may diminish over time.

Therefore, alternatives to these therapies are needed, which provide more significant reductions in blood pressure, persist indefinitely and which are safer, simpler, and less time-consuming.

### BRIEF SUMMARY OF THE INVENTION

The system and method of pre-aortic ganglion cell ablation offers a new effective method of controlling blood pressure in patients with medication resistant hypertension. It also overcomes the shortcomings of renal artery denervation. The present invention provides a system and method for ablating cell bodies within the pre-aortic ganglia for the treatment of hypertension. These ganglionic cells can be accessed endovascularly through the aorta itself, or through the celiac or superior mesenteric arteries. These methods of treating hypertension have not been previously described.

In one aspect of the invention a method of modulating a physiological parameter of a patient is provided, the method including disabling one or more pre-aortic ganglion cells within a pre-aortic ganglion and improving said physiological parameter.

In a further aspect of the invention, a method of modulating a physiological parameter of a patient is provided the method including destroying a pre-aortic ganglion cell to prevent regeneration.

In a further aspect of the invention, a method of modulating a physiological parameter of a patient is provided, the method including denervating one or more cells within a pre-aortic ganglion and improving said physiological parameter such that vessel spasm and dissection are avoided.

In a further aspect of the invention, a method of modulating a physiological parameter of a patient is provided, the method including denervating one or more cells within a pre-aortic ganglion and improving said physiological parameter such that deterioration of renal function is avoided.

In further aspects the invention is disclosed in the following numbered clauses:

1. A method for treating hypertension in a patient in need thereof, comprising:
providing an ablation device comprising an elongate member having a proximal end, a distal end, and proximal and distal regions, the elongate member also having at least one electrode located in the distal region;
inserting an introducer into the patient's paravertebral tissues and advancing the introducer through muscle at an anterior border of a vertebral body, the introducer comprising an elongate tubular member having a proximal end and a sharp distal end;
inserting the ablation device into the lumen of the introducer and advancing the elongate member past the vertebral body to the lateral wall of the aorta;
positioning the electrode at a region of interest adjacent a pre-aortic ganglia; and
activating the electrode to ablate cell bodies of the pre-aortic ganglia.

2. The method of clause 1, wherein the step of ablating irreversibly disables a plurality of cell bodies of the pre-aortic ganglia.

3. The method of clause 1, wherein the step of ablating permanently reduces the patient's hypertension.

4. The method of clause 1, wherein the elongate member carries more than one electrode.

5. The method of clause 1, wherein the elongate member carries one or more temperature sensor.

6. The method of clause 1, wherein the step of positioning the electrode is assisted by imaging.

7. The method of clause 1, wherein the distal end of the ablation device is atraumatic.

8. The method of clause 1, wherein the proximal end of the ablation device further comprises a handle.

9. The method of clause 1, wherein the at least one electrode emits radiation selected from the group consisting of radio frequency, microwave, and ultrasound.

10. The method of clause 1, wherein the elongate member is substantially rigid.

11. The method of clause 1, wherein the elongate member is about 15-25 gauge.

12. The method of clause 11, wherein the elongate member is about 22 gauge

13. The method of clause 1, wherein the elongate member defines a lumen and port for injection of pharmaceutical agent.

14. The method of clause 13, wherein the pharmaceutical agent is selected from the group consisting of an anesthetic or neurolytic.

15. The method of clause 1, wherein the ablation device further comprises wires extending from the at least one electrode and at least one temperature sensor located in the distal region to the proximal region of the elongate member.

16. The method of clause 1, wherein the elongate member has a curved distal end.

17. The method of clause 1, wherein the electrode is exposed to tissue on only one side of the elongate member.

18. The method of clause 1, further comprising the steps of repositioning the electrode at a second region of interest adjacent the pre-aortic ganglia and reactivating the electrode to ablate cell bodies of the pre-aortic ganglia.

19. The method of clause 1, wherein the electrode is activated for about 90 seconds.

20. The method of clause 1, further comprising the step of extending the electrode distally from the distal end of the introducer.

21. The method of clause 20, wherein the electrode fans out to provide a surface area greater than its surface area when in the collapsed state within the introducer.

22. The method of clause 21, wherein the electrode is a mesh

23. The method of clause 22, wherein the mesh is nitinol

24. The method of clause 22, wherein the mesh contains a plurality of electrodes.

25. The method of clause 20, wherein the electrode is carried on the surface of an elongate inflatable balloon.

26. The method of clause 25, further comprising the step of inflating the balloon.

27. The method of clause 1, wherein the electrode has a length of about 0.5 cm to 1.5 cm.

28. The method of clause 1, wherein the hypertension is resistant hypertension.

29. The method of clause 1, wherein the electrode is activated for between 60 and 120 seconds.

30. The method of clause 1, wherein activating said electrode causes heating of the cell bodies of the pre-aortic ganglia to between 60 and 70 degrees Celsius.

31. The method of clause 1, wherein positioning the electrode at a region of interest adjacent a pre-aortic ganglia is within 0 to 5 millimeters away from said ganglia.

32. The method of clause 1, wherein activation of electrode is done multiple times at multiple locations adjacent said ganglia.

33. A method for treating resistant hypertension in a patient in need thereof, comprising:
providing a cryoablation device comprising an elongate member having a proximal end, a distal end, proximal and distal regions, and a lumen extending from the proximal region to an orifice and an expansion chamber in the distal region, the elongate member also having at least one temperature sensor located in the distal region;
inserting an introducer into the patient's paravertebral tissues and advancing the introducer through muscle at an anterior border of a vertebral body, the introducer comprising an elongate tubular member having a proximal end and a sharp distal end;
inserting the cryoablation device into the lumen of the introducer and advancing the elongate member past the vertebral body to the lateral wall of the aorta;
positioning the expansion chamber at a region of interest adjacent a pre-aortic ganglia; and
infusing high pressure gas into the lumen of the cryoablation device to create an iceball and ablate cell bodies of the pre-aortic ganglia.

34. The method of clause 33, wherein the elongate member carries more than one temperature sensor.

35. The method of clause 33, wherein the step of positioning the expansion chamber is assisted by imaging.

36. The method of clause 33, wherein the distal end of the ablation device is atraumatic.

37. The method of clause 33, wherein the proximal end of the ablation device further comprises a handle.

38. The method of clause 33, wherein the elongate member is substantially rigid.

39. The method of clause 33, wherein the elongate member is about 15-25 gauge.

40. The method of clause 33, wherein the elongate member defines a second lumen and port for injection of a pharmaceutical agent.

41. The method of clause 40, wherein the pharmaceutical agent is selected from the group consisting of an anesthetic or neurolytic.

42. The method of clause 33, wherein the hypertension is resistant hypertension.

### BRIEF DESCRIPTION OF THE DRAWINGS

For a better understanding of the invention, and to show how the same may be carried into effect, reference will now be made, by way of example, to the accompanying drawings, in which:

FIG. 1 depicts Star-shaped meshwork of sympathetic cell bodies within the pre-aortic ganglia, positioned antero-lateral to the aortic wall and closely adherent to it.

FIG. 2 is a three-dimensional reconstruction of a human aorta showing the position of the celiac and superior mesenteric arteries.

FIG. 3 is an illustration showing the relationship between the right and left pre-aortic ganglia and the aorta.

FIG. 4 is a perspective view of a device in accordance with the invention for performing the method in accordance with the invention.

### DETAILED DESCRIPTION OF THE INVENTION

The present invention covers a system and method of ablating a portion of the cell bodies within the pre-aortic ganglia for the treatment of hypertension. These cells can be accessed endovascularly through the aorta itself, or through the celiac or superior mesenteric arteries. The systems and methods of treating hypertension in accordance with the invention have not been previously described.

Afferent sensory nerve fibers from the kidney, the adrenal and the renal artery itself enter the spinal cord through the dorsal root ganglion. They ascend in the spinal cord to the plethora of autonomic control centers within the brain and brainstem. Efferent sympathetic fibers destined for the kidney and adrenal glands, descend in the lateral column of the spinal cord and exit through the ventral root at each spinal level bilaterally. They traverse the white ramus communicantis and the sympathetic paraspinal ganglia in the lower thoracic and upper lumbar spine, and communicate with neighboring paraspinal sympathetic ganglia before they leave the paraspinal space. Pre-ganglionic segmental nerves from T6 to L1, mostly from T8 to T11, then circumnavigate the aorta, terminating at ganglionic cell bodies within the pre-aortic ganglia, namely the splanchnic, mesenteric, celiac, aortico-renal and suprarenal ganglia. Post-ganglionic fibres then track the vasculature, ultimately reaching the renal and adrenal arteries.

Surgical sympathetic denervation for the treatment of resistant hypertension was routinely performed in the 1940's. Such procedures involved removing various combinations of stellate ganglia in the neck, thoraco-lumbar paraspinal sympathetic ganglia, as well as stripping the aortic ganglia. Blood pressure decreases were very significant, and frequently associated with marked postural hypotension. Such surgical procedures were also associated with significant procedural morbidity and mortality, and were rapidly abandoned in favor of pharmacologic treatments which became available in the 1950's. Beta-blockers were followed by ACE inhibitors and other classes of anti-hypertensive medications. Pharmacotherapy became the mainstay of management for hypertensive patients during the second half of the last century. Many patients required more than one medication for adequate control of pressure, and up to a quarter of all remained hypertensive on multiple medications (resistant hypertension).

Recently mechanical means of controlling blood pressure have been revisited, specifically for patients with resistant hypertension. Renal artery denervation (RAD) involves ablating renal nerve fibres surrounding renal arteries bilaterally. The procedure involves advancing a catheter endovascularly into each of the renal arteries, and applying ablative energy through the wall of the artery to destroy some of the renal nerve fibres. The treatment lasts about 40 minutes. Procedure related complications are not uncommon. They include embolization from atheromatous renal arteries to kidneys whose function may already be impaired by chronic hypertension, and renal artery spasm or dissection which may also cause deterioration in renal function. As for efficacy, the procedure is moderately effective. While both systolic and diastolic pressures improve following this treatment, the longer term effect on blood pressure is as yet unknown. Peripheral nerve fibres such as those within the renal nerve typically regenerate, usually at the rate of 1 mm/month. Such regeneration following radiofrequency ablation has been frequently demonstrated (quote abstract). After a significant portion of ablated fibres regenerate, the beneficial effect of the procedure on blood pressure may be lost.

In this invention, we teach that denervation of cell bodies rather than nerve fibres may resolve the shortcomings of renal nerve denervation, and furthermore simplify the procedure itself while reducing complications attributable to the procedure. Regenerative capacity of ganglionic cell bodies is far less than that of nerve fibres. Thus following an ablative procedure, destroyed cell bodies will not recover from the insult and are replaced by glial tissue. Any reduction of blood pressure attributable to the procedure is thus likely to be permanent.

One method of denervating these cell bodies in accordance with the invention includes positioning an ablation device within an aorta of a patient, and advancing it to the level of the superior mesenteric artery or celiac artery, several centimeters above the take-off of the renal arteries. The ganglia are adherent to the antero-lateral aspects of the aorta and lie roughly 0.6 cm below the take-off of the celiac artery on the right and 0.9 cm below the same structure on the left. They can be up to 2.5 cm in length, and are organized somatotopically. These cell bodies are closely adherent to the antero-lateral aortic wall. In an alternative method, the ablation catheter could also be placed within the superior or inferior mesenteric arteries or celiac arteries rather than in the aorta itself. The relevant arteries could be localized angiographically, by ultrasound or by CT/MRI.

The ablation itself could be performed chemically, using pharmacologic agents or heat or cold, by using electric energy or electromagnetic energy such as radiofrequency or ultrasound, including high frequency focused ultrasound and low frequency ultrasound or any other technique that would destroy or partially destroy these structures for the treatment of hypertension. Several parameters may be used to determine further the exact localization of the pre-aortic sympathetic ganglia. By way of example, an energy delivery device may be provided to electrically stimulate the preganglionic fiber endings at the level of the ganglia might be associated with intercostal muscle twitching or contraction or be associated with pain or flushing in the relevant dermatome. Those of skill in the art will appreciate that other similar modes of stimulation may be used and that the energy delivery device may be configured to stimulate or ablate tissue. Lastly, changes in arterial pressure may occur. After the ganglia are localized, the mode may be switched from electrical stimulation to radiofrequency ultrasonic ablation and other modes known to those of skill in the art. Initially, this might cause blood pressure to increase or decrease abruptly. To prevent significant and sudden changes during the procedure and to be able to continuously monitor blood pressure, a pressure sensor may be added to the energy delivery device. The pressure sensor may be configured to feed information back to the energy delivery device and switch it off if blood pressure increases or decreases more than a predetermined amount.

The most significant benefit of this procedure, as already stated, is the extent and permanence of the hypotension achieved because ganglion cell bodies do not regenerate whereas nerve fibres do. In addition, the inventors have found that this method of treating hypertension is safer, simpler and less time-consuming than endovascular renal nerve ablation. The aorta is a huge structure easy to access, whereas renal arteries are smaller and not uncommonly stenosed in this population. Furthermore, dislodgement of atheromatous material from the wall of the aorta is unlikely to embolize to the kidneys and more likely to embolize to the lower limbs, sparing the kidneys. Vasospasm and renal artery dissection are not an issue with procedures being performed in the aorta, while they are very common during instrumentation of the renal artery. Furthermore, while renal nerve denervation involves treating both renal arteries, accessing the pre-aortic ganglia consists of a single procedure.

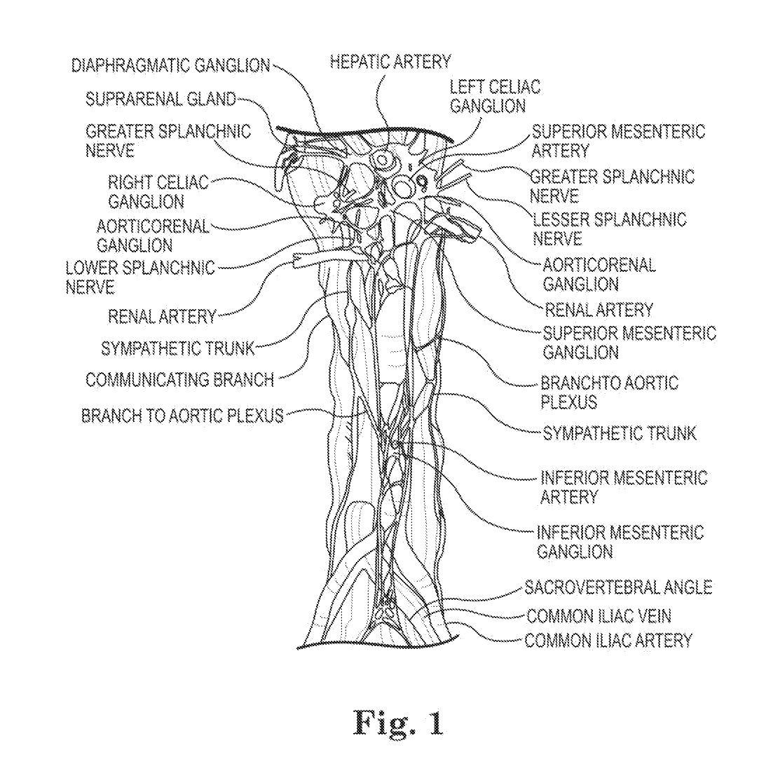

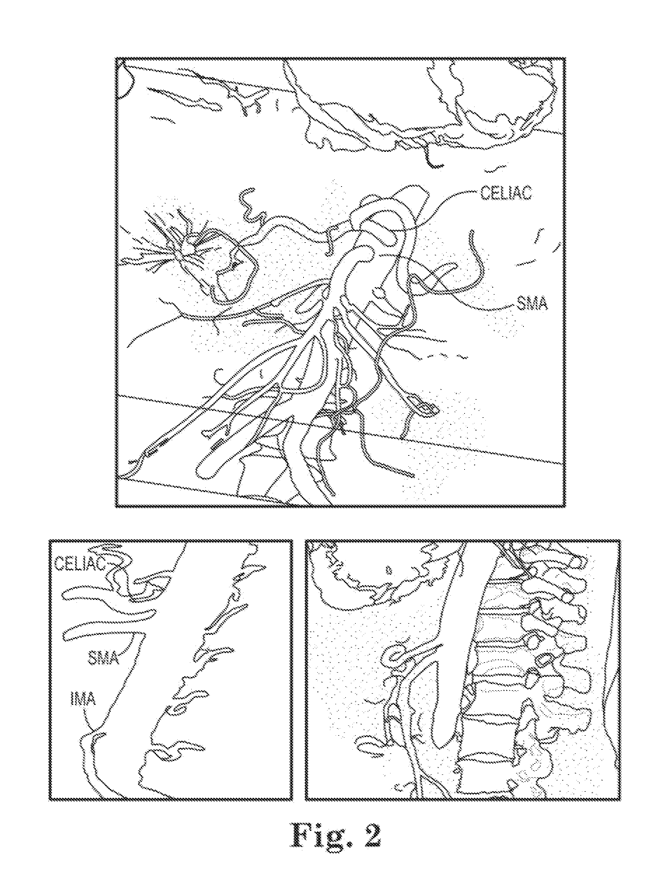

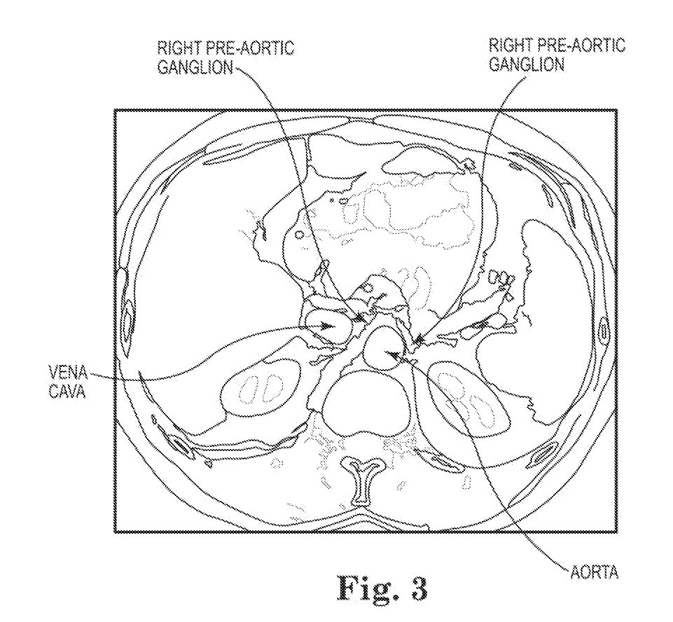

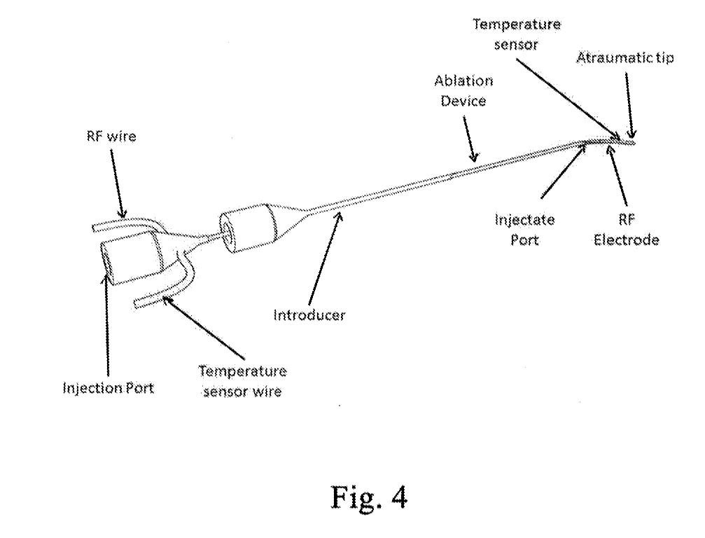

Referring now to FIGS. 1-4 the device and method in accordance with the invention will now be described. Referring to FIG. 4 the ablation device in accordance with the invention broadly includes an elongate member having a proximal end, a distal end, and proximal and distal regions, the elongate member also includes at least one electrode in the distal region. An introducer is also provided. The introducer includes an elongate tubular member having a proximal end and a sharp distal end. In operation, the introducer is inserted into the patient's paravertebral tissues and advanced through muscle at an anterior border of a vertebral body. The ablation device is inserted into the lumen of the introducer and the elongate body is advanced past the vertebral body to the lateral wall of the aorta. The electrode is positioned at a region of interest adjacent a pre-aortic ganglia and activated. The electrode thus ablates cell bodies of the pre-aortic ganglia.

Although the present invention has been described with reference to preferred embodiments, workers skilled in the art will recognize that changes may be made in form and detail without departing from the spirit and scope of the invention.

We claim:

1. A method for treating hypertension in a patient in need thereof, comprising:
providing an ablation device comprising an elongate member having a proximal end, a distal end, and proximal and distal regions, the elongate member also having at least one electrode located in the distal region;
inserting an introducer into the patient's paravertebral tissues and advancing the introducer through muscle at an anterior border of a vertebral body, the introducer comprising an elongate tubular member having a proximal end and a sharp distal end;
inserting the ablation device into the lumen of the introducer and advancing the elongate member past the vertebral body to the lateral wall of the aorta;
positioning the electrode at a region of interest adjacent a pre-aortic ganglia; and
activating the electrode to ablate cell bodies of the pre-aortic ganglia.

2. The method of claim 1, wherein the step of ablating irreversibly disables a plurality of cell bodies of the pre-aortic ganglia.

3. The method of claim 1, wherein the step of ablating permanently reduces the patient's hypertension.

4. The method of claim 1, wherein the elongate member carries more than one electrode.

5. The method of claim 1, wherein the elongate member carries one or more temperature sensor.

6. The method of claim 1, wherein the step of positioning the electrode is assisted by imaging.

7. The method of claim 1, wherein the distal end of the ablation device is atraumatic.

8. The method of claim 1, wherein the proximal end of the ablation device further comprises a handle.

9. The method of claim 1, wherein the at least one electrode emits radiation selected from the group consisting of radio frequency, microwave, and ultrasound.

10. The method of claim 1, wherein the elongate member is substantially rigid.

11. The method of claim 1, wherein the elongate member is about 15-25 gauge.

12. The method of claim 11, wherein the elongate member is about 22 gauge

13. The method of claim 1, wherein the elongate member defines a lumen and port for injection of pharmaceutical agent.

14. The method of claim 13, wherein the pharmaceutical agent is selected from the group consisting of an anesthetic or neurolytic.

15. The method of claim 1, wherein the ablation device further comprises wires extending from the at least one electrode and at least one temperature sensor located in the distal region to the proximal region of the elongate member.

16. The method of claim 1, wherein the elongate member has a curved distal end.

17. The method of claim 1, wherein the electrode is exposed to tissue on only one side of the elongate member.

18. The method of claim 1, further comprising the steps of repositioning the electrode at a second region of interest adjacent the pre-aortic ganglia and reactivating the electrode to ablate cell bodies of the pre-aortic ganglia.

19. The method of claim 1, wherein the electrode is activated for about 90 seconds.

20. The method of claim 1, further comprising the step of extending the electrode distally from the distal end of the introducer.

21. The method of claim 20, wherein the electrode fans out to provide a surface area greater than its surface area when in the collapsed state within the introducer.

22. The method of claim 21, wherein the electrode is a mesh

23. The method of claim 22, wherein the mesh is nitinol

24. The method of claim 22, wherein the mesh contains a plurality of electrodes.

25. The method of claim 20, wherein the electrode is carried on the surface of an elongate inflatable balloon.

26. The method of claim 25, further comprising the step of inflating the balloon.

27. The method of claim 1, wherein the electrode has a length of about 0.5 cm to 1.5 cm.

28. The method of claim 1, wherein the hypertension is resistant hypertension.

29. The method of claim 1, wherein the electrode is activated for between 60 and 120 seconds.

30. The method of claim 1, wherein activating said electrode causes heating of the cell bodies of the pre-aortic ganglia to between 60 and 70 degrees Celsius.

31. The method of claim 1, wherein positioning the electrode at a region of interest adjacent a pre-aortic ganglia is within 0 to 5 millimeters away from said ganglia.

32. The method of claim 1, wherein activation of electrode is done multiple times at multiple locations adjacent said ganglia.

33. A method for treating resistant hypertension in a patient in need thereof, comprising:
providing a cryoablation device comprising an elongate member having a proximal end, a distal end, proximal and distal regions, and a lumen extending from the proximal region to an orifice and an expansion chamber in the distal region, the elongate member also having at least one temperature sensor located in the distal region;
inserting an introducer into the patient's paravertebral tissues and advancing the introducer through muscle at an anterior border of a vertebral body, the introducer comprising an elongate tubular member having a proximal end and a sharp distal end;
inserting the cryoablation device into the lumen of the introducer and advancing the elongate member past the vertebral body to the lateral wall of the aorta;
positioning the expansion chamber at a region of interest adjacent a pre-aortic ganglia; and
infusing high pressure gas into the lumen of the cryoablation device to create an iceball and ablate cell bodies of the pre-aortic ganglia.

34. The method of claim 33, wherein the elongate member carries more than one temperature sensor.

35. The method of claim 33, wherein the step of positioning the expansion chamber is assisted by imaging.

36. The method of claim 33, wherein the distal end of the ablation device is atraumatic.

37. The method of claim 33, wherein the proximal end of the ablation device further comprises a handle.

38. The method of claim 33, wherein the elongate member is substantially rigid.

39. The method of claim 33, wherein the elongate member is about 15-25 gauge.

40. The method of claim 33, wherein the elongate member defines a second lumen and port for injection of a pharmaceutical agent.

41. The method of claim 40, wherein the pharmaceutical agent is selected from the group consisting of an anesthetic or neurolytic.

42. The method of claim 33, wherein the hypertension is resistant hypertension.
